SEQUENCE LISTING (30 sequences)

SEQ ID NO: 1 - protein, 15 aa, Artificial Sequence
  peptide; amino acid residues 12-26 of human p53 protein
PPLSQETFSDLWKLL

SEQ ID NO: 2 - protein, 9 aa, Artificial Sequence
  peptide; amino acid residues 12-20 of human p53 protein
PPLSQETFS

SEQ ID NO: 3 - protein, 10 aa, Artificial Sequence
  peptide; amino acid residues 17-26 of human p53 protein
ETFSDLWKLL

SEQ ID NO: 4 - protein, 17 aa, Artificial Sequence
  peptide; penetratin leader sequence from antennapedia
KKWKMRRNQFWVKVQRG

SEQ ID NO: 5 - protein, 14 aa, Artificial Sequence
  peptide; HIV-1 TAT membrane penetrating leader sequence
YGRKKRRQRRRPPQ

SEQ ID NO: 6 - protein, 13 aa, Artificial Sequence
  peptide; D-TAT membrane penetrating leader sequence
GRKKRRQRRRPPQ

SEQ ID NO: 7 - protein, 13 aa, Artificial Sequence
  peptide; R-TAT membrane penetrating leader sequence
GRRRRRRRRRPPQ

SEQ ID NO: 8 - protein, 7 aa, Artificial Sequence
  peptide; SV40-NLS membrane penetrating leader sequence
PKKKRKV

SEQ ID NO: 9 - protein, 16 aa, Artificial Sequence
  peptide; nucleoplasm-NLS membrane penetrating leader sequence
KRPAAIKKAGQAKKKK

SEQ ID NO: 10 - protein, 17 aa, Artificial Sequence
  peptide; HIV REV membrane penetrating leader sequence
TRQARRNRRRRWRERQR

SEQ ID NO: 11 - protein, 15 aa, Artificial Sequence
  peptide; FHV coat protein membrane penetrating leader sequence
RRRRNRTRRNRRRVR

SEQ ID NO: 12 - protein, 19 aa, Artificial Sequence
  peptide; BMV GAG membrane penetrating leader sequence
KMTRAQRRAAARRNRWTAR

SEQ ID NO: 13 - protein, 13 aa, Artificial Sequence
  peptide; HTLV-II REX membrane penetrating leader sequence
TRRQRTRRARRNR

SEQ ID NO: 14 - protein, 19 aa, Artificial Sequence
  peptide; CCMV GAG membrane penetrating leader sequence
KLTRAQRRAAARKNKRNTR

SEQ ID NO: 15 - protein, 17 aa, Artificial Sequence
  peptide; P22 N membrane penetrating leader sequence
NAKTRRHERRRKLAIER

SEQ ID NO: 16 - protein, 22 aa, Artificial Sequence
  peptide; LAMBDA N membrane penetrating leader sequence
MDAQTRRRERRAEKQAQWKAAN

SEQ ID NO: 17 - protein, 18 aa, Artificial Sequence
  peptide; Phi N membrane penetrating leader sequence
TAKTRYKARRAELIAERR

SEQ ID NO: 18 - protein, 16 aa, Artificial Sequence
  peptide; Yeast PRP6 membrane penetrating leader sequence
TRRNKRNRIQEQLNRK

SEQ ID NO: 19 - protein, 12 aa, Artificial Sequence
  peptide; Human U2AF membrane penetrating leader sequence
SQMTRQARRLYV

SEQ ID NO: 20 - protein, 26 aa, Artificial Sequence
  peptide; Human C-FOS membrane penetrating leader sequence
KRRIRRERNKMAAAKSRNRRRELTDT

SEQ ID NO: 21 - protein, 28 aa, Artificial Sequence
  peptide; Human C-JUN membrane penetrating leader sequence
RIKAERKRMRNRIAASKSRKRKLERIAR

SEQ ID NO: 22 - protein, 22 aa, Artificial Sequence
  peptide; Yeast GCN4 membrane penetrating leader sequence
KRARNTEAARRSRARKLQRMKQ

SEQ ID NO: 23 - protein, 18 aa, Artificial Sequence
  peptide; membrane penetrating leader sequence
KLALKLALKALKAALKLA

SEQ ID NO: 24 - protein, 18 aa, Artificial Sequence
  peptide; p-vec membrane penetrating leader sequence
LLIILRRRIRKQAKAHSK

SEQ ID NO: 25 - protein, 13 aa, Artificial Sequence
  peptide; used as a control
MPFSTGKRIMLGE

SEQ ID NO: 26 - protein, 8 aa, Artificial Sequence
  peptide; Arg(8) membrane penetrating leader sequence
RRRRRRRR

SEQ ID NO: 27 - protein, 16 aa, Artificial Sequence
  peptide; poly-R membrane penetrating sequence
RRRRXXXXXXXXXXXX

SEQ ID NO: 28 - protein, 15 aa, Artificial Sequence
  peptide; retro-inverso peptide analog of SEQ ID NO1; all D-amino acids
LLKWLDSFTEQSLPP

SEQ ID NO: 29 - protein, 9 aa, Artificial Sequence
  peptide; retro-inverso peptide analog of SEQ ID NO2; all D-amino acids
SFTEQSLPP

SEQ ID NO: 30 - protein, 10 aa, Artificial Sequence
  peptide; retro-inverso peptide analog of SEQ ID NO3; all D-amino acids
LLKWLDSFTE

(19) United States
(12) Patent Application Publication
(10) Pub. No.: US 2005/0245451 A1
(43) Pub. Date: Nov. 3, 2005

(54) Peptides selectively lethal to malignant and transformed mammalian cells

(76) Inventor: Matthew R. Pincus, Brooklyn, NY (US)

Correspondence Address: DILWORTH & BARRESE, LLP, 333 EARLE OVINGTON BLVD., UNIONDALE, NY, 11553, US

(21) Appl. No.: 11/019,894

(22) Filed: Dec. 21, 2004

Related U.S. Application Data
(63) Continuation-in-part of application No. 10/386,737, filed on Mar. 12, 2003.
Application No. 10/386,737 is a continuation-in-part of application No. 09/827,683, filed on Apr. 5, 2001, now abandoned.
(60) Provisional application No. 60/363,785, filed on Mar. 12, 2002.
(60) Provisional application No. 60/195,102, filed on Apr. 5, 2000.

(51) Int. Cl. A61K 38/10; A61K 38/08; C07K 7/08

(52) U.S. Cl. 514/13; 514/14; 514/15; 530/326; 530/327; 530/328

(57) ABSTRACT

The present invention provides peptides corresponding to all or a portion of amino acid residues 12-26 of human p53 protein, which peptides are lethal to malignant or transformed cells when fused to a membrane-penetrating leader sequence. In order to reduce proteolysis of a subject peptide, one or more D-amino acids may be substituted for the corresponding L-amino acids in the p53 portion and/or the membrane-penetrating leader of a subject peptide. Further, a pseudopeptide bond or a retro-inverso pseudopeptide bond may be substituted for one or more peptide bonds in either or both of the p53 sequence or membrane-penetrating leader sequence in order to render a subject peptide less susceptible to proteolysis. In addition, both the membrane-penetrating leader sequence and the p53 portion of a subject peptide may comprise retro-inverso, and partially modified retro-inverso isomers. Such isomers are less susceptible to proteolysis and therefore have prolonged half-lives. The subject peptides are useful in treating neoplastic disease in an animal, preferably a human. Also provided are pharmaceutical compositions comprising the subject peptides admixed with a pharmaceutical acceptable carrier. Methods of treating neoplastic disease in a patient by administering a subject peptide fused at its carboxy terminal end to a membrane-penetrating leader sequence are also provided as are methods of assessing the level of effectiveness of a subject peptide in killing malignant, transformed, or neoplastic cells in vitro.

This application is a continuation-in-part application of application Ser. No. 10/386,737, filed Mar. 12, 2003 which is a continuation-in-part of application Ser. No. 09/827,683, filed Apr. 5, 2001; U.S. Ser. No. 10/386,737 claims the benefit of U.S. Provisional Application No. 60/363,785, filed Mar. 12, 2002, and U.S. Ser. No. 09/827,683 claims the benefit of U.S. Provisional Application Ser. No. 60/195,102, filed Apr. 5, 2000.

### BACKGROUND OF THE INVENTION

This invention relates to therapeutic modalities for treatment of neoplastic disease. More specifically, this invention involves synthetic peptides that selectively destroy malignant and transformed cells, and a method for treatment of neoplastic disease based thereon.

The p53 protein is a vital regulator of the cell cycle. It blocks the oncogenic effects of a number of oncogene proteins that induce mitosis, in part by blocking transcription of proteins that induce mitosis and by inducing the transcription of proteins that block mitosis, and promote apoptosis. Absence of the p53 protein is associated with cell transformation and malignant disease. Haffner, R & Oren, M. (1995) Curr. Opin. Genet. Dev. 5: 84-90.

The p53 protein molecule consists of 393 amino acids. It includes domains that bind to specific sequences of DNA in a DNA-binding domain that consists of residues 93-313. The crystal structure of this region has been determined by x-ray crystallography. Residues 312-393 are involved in the formation of homotetramers of the p53 protein. Residues 1-93 are involved in regulation of the activity and half life of the p53 protein.

The p53 protein binds to another important regulatory protein, the MDM-2 protein. The MDM-gene that encodes the MDM-2 protein is a known oncogene. The MDM-2 protein forms a complex with the p53 protein, which results in the degradation of the p53 protein by a ubiquination pathway. The p53 protein binds to MDM-2 protein using an amino acid sequence that includes residues 14-22 of the p53 protein, which are invariant. The entire MDM-2 protein binding domain of the p53 protein spans residues 12-26. Haffner, R & Oren, M. (1995) Curr. Opin. Genet. Dev. 5: 84-90.

Considering that the MDM-2 protein is the expression product of a known oncogene, it is not surprising that MDM-2 protein is a very important regulatory protein. Moreover, overexpression or amplification of MDM-2 protein has been found in 40-60% of human malignancies, including 50% of human breast tumors. It has been suggested that formation of a complex between the p53 protein and the MDM-2 protein may result in the inhibition of transcription activity of the p53 protein, and thus the anti-tumor effect of the molecule by blocking of an activation domain of the p53 protein, or of a DNA binding site within it. More generally, these and other experimental observations have been interpreted as suggesting that the anti-tumor effect of the p53 protein might be enhanced by peptides capable of interfering with the binding of the MDM-2 protein to the p53 protein. Indeed, a number of investigators have suggested that the MDM-2/p53 complex might be a target for rational drug design. See, e.g., Christine Wasylyk et al., “p53 Mediated Death of Cells Overexpressing MDM2 by an Inhibitor of MDM2 Interaction with p53”, Oncogene, 18, 1921-34 (1999), and U.S. Pat. No. 5,770,377 to Picksley et al.

Evolution has ensured the almost exclusive occurrence of L-amino acids in naturally occurring proteins. Virtually all proteases therefore cleave peptide bonds between adjacent L-amino acids; thus, artificial proteins or peptides composed of D-amino acids are largely resistant to proteolytic breakdown. See, e.g., U.S. patent application Ser. No. 10/399,127. Serum proteases have specific substrate requirements. In order for proteases to cleave, the substrate must have both L-amino acids and peptide bonds (Power et al. (1993 Pharmaceutical Res. 10:1268-1273).

Linear modified retro-peptide structures appear in the literature and the term “retro-isomer” was designated to include an isomer in which the direction of the sequence is reversed compared with the parent peptide. See, e.g., Goodman, M., et al., “On the Concept of Linear Modified Retro-Peptide Structures”, Acc. of Chem. Res., 12(1), 1-7 (1979) and U.S. patent application Ser. No. 10/399,127 to Bonny. Retro-inverso isomers in which the direction of the sequence is reversed and the chirality of each amino acid residue is inverted also appear in the literature.

Recently, Jameson et al. reportedly engineered an analogue of the hairpin loop of the CD4 receptor by combining these two properties: reverse synthesis and a change in chirality. See, e.g., Jameson et al., “A rationally designed CD4 analogue inhibits experimental allergic encephalomyelitis”, Nature, 368, 744-746 (1994) and Brady, L. et al., “Reflections on a Peptide”, Nature, 368, 692-693 (1994). The net result of combining D-enantiomers and reverse synthesis is that the positions of carbonyl and amino groups in each amide bond are exchanged, while the position of the side-chain groups at each alpha carbon is preserved. Jameson et al. reportedly demonstrated an increase in biological activity for their reverse D peptide, which contrasts the limited in vivo activity of its conventional all-L enantiomer, owing to its susceptibility to proteolysis.

### SUMMARY OF THE INVENTION

The present invention provides a peptide comprising at least about six contiguous amino acids of the amino acid sequence: PPLSQETFSDLWKLL (SEQ ID NO:1), or an analog or derivative thereof, wherein said peptide or analog or derivative thereof is fused to a membrane-penetrating leader sequence and is selectively lethal to malignant or transformed cells.

Examples of such peptides include PPLSQETFSDLWKLL (SEQ ID NO:1) or an analog or derivative thereof, PPLSQETFS (SEQ ID NO:2) or an analog or derivative thereof and ETFSDLWKLL (SEQ ID NO:3) or an analog or derivative thereof. In order to be transported across a cell membrane and selectively kill a malignant or transformed cell, the leader sequence is preferably positioned at the carboxyl terminal end of the peptide, analog, or derivative thereof. Preferably, the leader sequence comprises predominantly positively charged amino acid residues. Examples of leader sequences which may be used in accordance with the present invention include but are not limited to penetratin (KKWKMRRNQFWVKVQRG)(SEQ ID NO:4); (Arg)₈ (SEQ ID NO:26) or any poly-R from (R)₄-(R)₁₆ (SEQ ID NO:27); HIV-1 TAT(47-60) (YGRKKRRQRRRPPQ)(SEQ ID NO:5); D-TAT (GRKKRRQRRRPPQ) (SEQ ID NO:6); R-TAT G(R)₉PPQ(SEQ ID NO:7); SV40-NLS (PKKKRKV)(SEQ ID NO:8); nucleoplasmin-NLS (KRPAAIKKAGQAKKKK)(SEQ ID NO:9); HIV REV (34-50)-(TRQARRNRRRRWRERQR)(SEQ ID NO:10); FHV (35-49) coat-(RRRRNRTRRNRRRVR)(SEQ ID NO:11); BMV GAG (7-25)-(KMTRAQRRAAARRNRWTAR)(SEQ ID NO:12); HTLV-II REX 4-16-(TRRQRTRRARRNR)(SEQ ID NO:13); CCMV GAG (7-25)-(KLTRAQRRAAARKNKRNTR)(SEQ ID NO:14); P22 N (14-30)(NAKTRRHERRRKLAIER)(SEQ ID NO:15); LAMBDA N (1-22)(MDAQTRRRERRAEKQAQWKAAN)(SEQ ID NO:16); Phi N (12-29) (TAKTRYKARRAELIAERR)(SEQ ID NO:17); YEAST PRP6 (129-124) (TRRNKRNRIQEQLNRK) (SEQ ID NO:18); HUMAN U2AF (SQMTRQARRLYV)(SEQ ID NO:19); HUMAN C-FOS (139-164) KRRIRRERNKMAAAKSRNRRRELTDT (SEQ ID NO:20); HUMAN C-JUN (252-279) (RIKAERKRMRNRIAASKSRKRKLERIAR)(SEQ ID NO:21); YEAST GCN4 (KRARNTEAARRSRARKLQRMKQ)(SEQ ID NO:22); KLALKLALKALKAALKLA(SEQ ID NO:23); p-vec LLIILRRRIRKQAKAHSK(SEQ ID NO:24). Preferably, the positively charged leader sequence of the penetration leader sequence of antennapedia protein is used.

The present invention further contemplates that any of the subject peptides described hereinabove may have specific alterations made thereto, which alterations render the peptides less susceptible to proteolysis. For example, a subject peptide may have one or more peptide bonds replaced with an isostere pseudopeptide bond or a retro-inverso pseudopeptide bond. In another embodiment of the invention, a subject peptide may be a directional peptide isomer of the corresponding portion of the naturally occurring p53 protein. In this embodiment of the invention, enantio, retro-inverso, and partially modified retro-inverso peptides are particularly contemplated.

Thus, the present invention provides a subject peptide, analog or derivative thereof, hereinbefore described, fused to a membrane-penetrating leader sequence, and selectively lethal to malignant or transformed cells, which comprises one or more D-amino acids, or which has at least one peptide bond substituted with an isostere pseudopeptide bond or a retro-inverso pseudopeptide bond.

For example, a D-amino acid may be positioned at the N-terminus of a subject peptide. The presence of an N-terminal D-amino acid increases the stability of a peptide since exopeptidases acting on the N-terminal residue cannot utilize a D-amino acid as substrate. In another embodiment, a D-amino acid may be positioned at the C-terminus of a subject peptide. The presence of a C-terminal D-amino acid also stabilizes the peptide since exopeptidases acting on the C-terminal residue cannot utilize a D-amino acid as a substrate.

In particular, there is provided a peptide comprising at least six contiguous amino acids of the amino acid sequence: PPLSQETFSDLWKLL (SEQ ID NO:1), or an analog or derivative thereof, wherein said peptide or analog or derivative thereof is fused to a membrane-penetrating leader sequence, is selectively lethal to malignant or transformed cells and wherein at least one amino acid is in the D form or wherein one or more peptide bonds are replace with an isostere pseudopeptide bond or a retro-inverso pseudopeptide bond. Examples include peptides comprising the amino acid sequence: PPLSQETFS (SEQ ID NO: 2), ETFSDLWKLL (SEQ ID NO: 3), or an analog or derivative thereof.

In another embodiment, a subject peptide, analog or derivative thereof, fused to a membrane-penetrating leader sequence and selectively lethal to malignant or transformed cells, comprises all D-amino acids assembled in reverse order to the natural sequence found in the p53 protein. Such peptide is referred to herein as a retro-inverso (RI) peptide.

For example, there is provided a retro-inverso (RI) peptide comprising at least six contiguous D-amino acids assembled in the reverse order of the amino acid sequence: PPLSQETFSDLWKLL (SEQ ID NO:1), or an analog or derivative thereof, fused to a membrane-penetrating leader sequence, and selectively lethal to malignant or transformed cells. In a preferred embodiment, the peptide comprises at least six contiguous D-amino acids assembled in the reverse order of the amino acid sequence PPLSQETFS (SEQ ID NO: 2) or ETFSDLWKLL (SEQ ID NO: 3), or an analog or derivative thereof.

A partially modified retro-inverso (PMRI) peptide is also provided wherein only a portion of the p53 sequence is retro-inverted. For example, there is provided a peptide comprising at least six amino acids having only a portion of amino acids in the D form and assembled in reverse order of the amino acid sequence set forth in SEQ ID NO:1. In a preferred embodiment, a portion of the at least six D-amino acids are assembled in reverse order of the sequence PPLSQETFS (SEQ ID NO: 2) or ETFSDLWKLL (SEQ ID NO: 3), or an analog or derivative thereof.

In any of the foregoing peptides comprising one or more D-amino acids or one or more isostere pseudopeptide bonds or inverso pseudopeptide bonds, or in any of the foregoing retro-inverso, or partially modified retro-inverso peptides, the membrane-penetrating leader sequence may also comprise one or more D-amino acids or one or more isostere pseudopeptide bonds or inverso pseudopeptide bonds.

In another embodiment of the invention, the membrane penetrating leader sequences are themselves retro-inverso, or partially modified retro-inverso peptide isomers. A membrane-penetrating leader sequence in a retro-inverso form comprises all D-amino acids assembled in reverse order to any of the sequences set forth in SEQ ID NOs: 4-24 or SEQ ID NOs: 26-27. A membrane penetrating leader sequence in a partially modified retro-inverso form has only a portion of the amino acid residues in a D-form and in reverse order to any of the sequences set forth in SEQ ID NOs: 4-24 or SEQ ID NOs: 26-27.

Pharmaceutical compositions comprising at least one of the subject peptides admixed with a pharmaceutically acceptable carrier are also provided. Such pharmaceutical compositions may also include any of the subject peptides comprising one or more D-amino acids or one or more isostere pseudopeptide bonds or retro-inverso pseudopeptide bonds, and may also include any of the subject retro-inverso, and partially modified retro-inverso peptides. In addition, methods for treating neoplastic disease in a subject i.e., selectively killing malignant or neoplastic cells in a subject, are provided. In one embodiment, the method comprises administering to the subject, a therapeutically effective amount of a peptide comprising at least about six contiguous amino acids of the amino acid sequence: PPLSQETFSDLWKLL (SEQ ID NO:1), or an analog or derivative thereof, wherein said peptide or analog or derivative thereof is fused at its carboxy terminal end to a membrane-penetrating leader sequence and is selectively lethal to malignant or transformed cells. In another embodiment, the method comprises administering to the subject, a therapeutically effective amount of at least one peptide having the sequence set forth in SEQ ID NO: 1, SEQ ID NO: 2, or SEQ ID NO: 3 or an analog or derivative thereof, wherein a membrane-penetrating leader sequence is fused to the carboxy terminal end of the peptide, analog, or derivative thereof.

Further embodiments of the method comprise administering to a subject a therapeutically effective amount of a subject peptide comprising one or more D-amino acids or one or more isostere pseudopeptide bonds or retro-inverso pseudopeptide bonds, or a retro-inverso, or partially modified retro-inverso peptide, or an analog or derivative thereof, wherein a membrane-penetrating leader sequence is fused to the carboxy terminal end of the peptide, analog, or derivative thereof.

Also provided are methods of assessing the effectiveness of a subject peptide in killing malignant, neoplastic, or transformed cells in vitro. The method comprises the steps of contacting malignant, transformed or neoplastic cells in vitro with at least one subject peptide or an analog or derivative thereof, and assessing the level of effectiveness of the peptide based on the ratio or percentage of dead cells compared to live cells and its effect on the growth of untransformed (normal) cells in culture.

### BRIEF DESCRIPTION OF THE DRAWINGS

FIG. 1 graphically depicts the in vivo tumor-inhibiting effect of PNC-28 (SEQ ID NO:3 fused at its carboxy terminal end to SEQ ID NO:4) in homozygous NU/NU mice xenotransplanted with pancreatic carcinoma cells. The arrow with a star indicates the time of s.c. pump implantation on day 13 (precisely 13.5) during the tumor growth period.

### DETAILED DESCRIPTION OF THE INVENTION

In accordance with the present invention, it has been discovered that malignant and transformed cells are selectively destroyed by administration of a synthetic peptide comprising a sequence of amino acids within the p53 protein and a leader sequence as a single continuous polypeptide chain. The mechanism of action appears to be independent of the p53 protein binding to the MDM-2 protein, as the p53 peptide selectively kills transformed cells that do not produce the p53 protein at all. The p53 peptide also selectively kills malignant and transformed cells that express normal or elevated levels of the p53 protein without killing normal cells.

In accordance with the present invention, there are provided compositions comprising peptides corresponding to all or a portion of amino acid residues 12-26 of human p53. This region is known to contact the mdm-2 protein and adopts an α-helical conformation when bound to mdm-2. When fused on the carboxy-terminal end with a membrane-penetrating leader sequence, the subject peptides selectively kill malignant and transformed human cells.

In a first aspect of the invention, there is provided a peptide comprising at least about six contiguous amino acids of the following amino acid sequence: PPLSQETFSDLWKLL (SEQ ID NO:1), wherein the peptide comprising at least about six contiguous amino acids is fused to a leader sequence. Preferably, the peptide comprises from at least about eight (8) to at least about fifteen (15) amino acid residues. In a preferred embodiment, a peptide comprising from at least about eight (8) to at least about 15 (fifteen) amino acids of the sequence set forth in SEQ ID NO:1 has the following amino acid sequence: PPLSQETFSDLWKLL (SEQ ID NO:1). In another preferred embodiment, a peptide comprising from at least about eight (8) to at least about 15 (fifteen) amino acids of the sequence set forth in SEQ ID NO:1 has the following amino acid sequence: PPLSQETFS (SEQ ID NO:2). In still another preferred embodiment, a peptide comprising from at least about eight (8) to at least about fifteen (15) amino acids of the sequence set forth in SEQ ID NO:1 has the following amino acid sequence: ETFSDLWKLL (SEQ ID NO:3).

Leader sequences which function to import the peptides of the invention into a cell may be derived from a variety of sources. Preferably, the leader sequence comprises predominantly positively charged amino acid residues since a positively charged leader sequence stabilizes the alpha helix of a subject peptide. Examples of leader sequences which may be linked to the peptides of the present invention are described in Futaki, S. et al (2001) Arginine-Rich Peptides, J. Biol. Chem. 276:5836-5840, and include but are not limited to the following membrane-penetrating leader sequences (numbering of the amino acid residues making up the leader sequence of the protein is indicated parenthetically immediately after the name of the protein in many cases): penetratin (KKWKMRRNQFWVKVQRG)(SEQ ID NO:4); (Arg)₈ (SEQ ID NO:26) or any poly-R from (R)₄-(R)₁₆ (SEQ ID NO:27); HIV-1 TAT(47-60) (YGRKKRRQRRRPPQ)(SEQ ID NO:5); D-TAT (GRKKRRQRRRPPQ) (SEQ ID NO:6); R-TAT G(R)₉PPQ(SEQ ID NO:7); SV40-NLS (PKKKRKV)(SEQ ID NO:8); nucleoplasmin-NLS (KRPAAIKKAGQAKKKK)(SEQ ID NO:9); HIV REV (34-50)-(TRQARRNRRRRWRERQR)(SEQ ID NO:10); FHV (35-49) coat-(RRRRNRTRRNRRRVR)(SEQ ID NO:11); BMV GAG (7-25)-(KMTRAQRRAAARRNRWTAR)(SEQ ID NO:12); HTLV-II REX 4-16-(TRRQRTRRARRNR)(SEQ ID NO:13); CCMV GAG (7-25)-(KLTRAQRRAAARKNKRNTR)(SEQ ID NO:14); P22 N (14-30)(NAKTRRHERRRKLAIER)(SEQ ID NO:15); LAMBDA N (1-22)(MDAQTRRRERRAEKQAQWKAAN)(SEQ ID NO:16); Phi N (12-29) (TAKTRYKARRAELIAERR)(SEQ ID NO:17); YEAST PRP6 (129-124) (TRRNKRNRIQEQLNRK) (SEQ ID NO:18); HUMAN U2AF (SQMTRQARRLYV)(SEQ ID NO:19); HUMAN C-FOS (139-164) KRRIRRERNKMAAAKSRNRRRELTDT (SEQ ID NO:20); HUMAN C-JUN (252-279) (RIKAERKRMRNRIAASKSRKRKLERIAR)(SEQ ID NO:21); YEAST GCN4 (KRARNTEAARRSRARKLQRMKQ)(SEQ ID NO:22); KLALKLALKALKAALKLA(SEQ ID NO:23); p-vec LLIILRRRIRKQAKAHSK(SEQ ID NO:24). Other membrane penetrating leader sequences may also be used. Such sequences are widely available and are described e.g., in Scheller et al. (2000) Eur. J. Biochem. 267:6043-6049, and Elmquist et al., (2001) Exp. Cell Res. 269:237-244.

Preferably, the positively charged leader sequence of the penetration leader sequence of antennapedia protein is used. This leader sequence has the following amino acid sequence: KKWKMRRNQFWVKVQRG (SEQ ID NO: 4). Preferably, the leader sequence is attached to the carboxyl terminal end of the p53 peptide to enable the synthetic peptide to kill transformed and malignant cells.

In order to reduce susceptibility to proteolytic degradation, a subject peptide hereinbefore described, i.e., any of SEQ ID NOs.: 1-3 (p53 peptides) or SEQ ID NOs: 4-25 or 26-27 (membrane-penetrating leader sequences) may comprise one or more amino acids in the D-form and/or may comprise one or more isostere pseudopeptide bonds or one or more retro-inverso pseudopeptide bonds. Thus for example, as little as one or as many as all amino acids of a subject peptide may be in the D form. Preferably, a D-amino acid is positioned at the N-terminus of a subject peptide. Such positioning renders the peptide less susceptible to exopeptidases that act on N-terminal residues since such exopeptidases cannot utilize a D-amino acid as a substrate. A D-amino acid may also be positioned at the C-terminus of the membrane-penetrating leader sequence, which sequence is fused to the p53 peptide at its carboxy terminus. Such positioning of a D-amino acid helps stabilize the peptide since exopeptidases acting on C-terminal residues cannot utilize D-amino acids as a substrate.

Alternatively, a subject peptide of the present invention can be synthesized as a retro-inverso peptide (RI) comprising all D-amino acids as well as a reversed sequence. In this embodiment, the peptide comprises both reversed sequence and inverted stereochemistry at all chiral centers.

In still another embodiment, a subject peptide may be synthesized as a partially modified retro-inverso peptide (PMRI) wherein only a portion of the p53 sequence or membrane-penetrating leader sequence is retro-inverted. For example, there is provided a peptide comprising at least six amino acids having only a portion of amino acids in the D form and assembled in reverse order.

In particular, there is provided a peptide comprising at least six contiguous amino acids of the amino acid sequence: PPLSQETFSDLWKLL (SEQ ID NO: 1), or an analog or derivative thereof, wherein said peptide or analog or derivative thereof is fused to a membrane-penetrating leader sequence, is selectively lethal to malignant or transformed cells and wherein at least one amino acid is in the D form or wherein one or more peptide bonds are replace with an isostere pseudopeptide bond or a retro-inverso pseudopeptide bond. Examples include peptides comprising the amino acid sequence: PPLSQETFS (SEQ ID NO: 2), ETFSDLWKLL (SEQ ID NO: 3), or an analog or derivative thereof.

In another embodiment, a subject peptide, analog or derivative thereof, fused to a membrane-penetrating leader sequence and selectively lethal to malignant or transformed cells, comprises all D-amino acids assembled in reverse order to the natural sequence found in the p53 protein. Such peptide is referred to herein as a retro-inverso (RI) peptide.

For example, there is provided a retro-inverso (RI) peptide comprising at least six contiguous D-amino acids assembled in the reverse order of the amino acid sequence: PPLSQETFSDLWKLL (SEQ ID NO: 1), or an analog or derivative thereof, fused to a membrane-penetrating leader sequence, and selectively lethal to malignant or transformed cells. In a preferred embodiment, the peptide comprises at least about six contiguous D-amino acids assembled in the reverse order of the amino acid sequence PPLSQETFS (SEQ ID NO: 2) or ETFSDLWKLL (SEQ ID NO: 3), or an analog or derivative thereof.

A partially modified retro-inverso (PMRI) peptide is also provided wherein only a portion of the p53 sequence is retro-inverted. For example, there is provided a peptide comprising at least six amino acids having only a portion of amino acids in the D form and assembled in reverse order of the amino acid sequence set forth in SEQ ID NO: 1. In a preferred embodiment, a portion of the at least six D-amino acids are assembled in reverse order of the sequence PPLSQETFS (SEQ ID NO: 2) or ETFSDLWKLL (SEQ ID NO: 3), or an analog or derivative thereof.

In any of the foregoing peptides comprising one or more D-amino acids or one or more isostere pseudopeptide bonds or inverso pseudopeptide bonds, or in any of the foregoing retro-inverso, or partially modified retro-inverso peptides, the membrane-penetrating leader sequence is located at the carboxyl terminal end of the peptide, analog or derivative thereof in order to cross the cell membrane and specifically kill malignant, transformed, or neoplastic cells. However, peptides having a membrane-penetrating leader sequence at the N-terminal end of the p53 peptide are useful as control peptides in various experiments such as the in vitro experiments described herein. Further, the membrane-penetrating leader sequence may also comprise one or more D-amino acids or one or more isostere pseudopeptide bonds or inverso pseudopeptide bonds.

In another embodiment of the invention, the membrane penetrating leader sequences are themselves retro-inverso, or partially modified retro-inverso peptide isomers. A membrane-penetrating leader sequence in a retro-inverso form comprises all D-amino acids assembled in reverse order to any of the sequences set forth in SEQ ID NO: 4-24 or SEQ ID NO:26-27. A membrane penetrating leader sequence in a partially modified retro-inverso form has only a portion of the amino acid residues in a D-form and in reverse order to any of the sequences set forth in SEQ ID NO: 4-24 or SEQ ID NO:26-27.

The synthesis of RI and PMRI peptides results in the introduction of two non-amino acid residues into the newly formed isomers, the gem-diaminoalkyl (gxaa) and the 2-alkylmalonyl (mXaa) residues at the amino and carboxy sides of the transformed sequence, respectively. See, e.g., Scheibler, L. and Chorev, M. (2003) In Synthesis of Peptides and Peptidomimetics (Houben-Weyl Methods of Organic Chemistry, 4^{th} Ed., Vol. 22C) (Goodman, M., ed), pp. 528-551, Thieme, Stuttgart, incorporated by reference herein as if fully set forth. As described in Fischer, P. M. (2003) “The Design, Synthesis and Application of Stereochemical and Directional Peptide Isomers: A Critical Review” Current Protein and Peptide Sequence 4: 339-356, stereochemical and directional peptide isomers that do not contain directional converter residues present no synthetic difficulties. Such peptides may be obtained using solid-phase peptide synthesis methods using appropriate N^{α}- and side chain-protected L and D amino acids. See Chan et. al., (2000) Fmoc Solid Phase Peptide Synthesis: a Practical Approach, Oxford University Press. Both Fischer, P. M. (2003) and Chan et al. (2000) are incorporated by reference herein as if fully set forth.

Synthesis of PMRI peptides however, is more difficult due to the different types and numbers of amino acid residues flanking the direction-reversing gxaa and mXaa residues. Fischer, P. M (2003) however, provides an applicable scheme for gxaa and mXaa monomer preparation as well as peptide assembly for use in synthesizing the PMRI peptides of the present invention.

Structurally related amino acid sequences may be substituted for the disclosed sequences set forth in SEQ ID NOs: 1, 2, 3, or 4 in practicing the present invention. Any of the sequences set forth in SEQ ID NOs: 1, 2 or 3, including analogues or derivatives thereof, when joined with a leader sequence, including, but not limited to the sequence set forth in SEQ ID NO: 4, will be referred to herein as either a “synthetic peptide” or “synthetic peptides.” Rigid molecules that mimic the three dimensional structure of these synthetic peptides are called peptidomimetics and are also included within the scope of this invention. Alpha helix stabilizing amino acid residues at either or both the amino or carboxyl terminal ends of the p53 peptide may be added to stabilize the alpha helical conformation which is known to be the conformation of this region of the p53 protein when it binds to the MDM-2 protein. Examples of alpha helical stabilizing amino acids include Leu, Glu (especially on the amino terminal of the helix), Met and Phe.

Amino acid insertional derivatives of the peptides of the present invention include amino and/or carboxyl terminal fusions as well as intra-sequence insertions of single or multiple amino acids. Insertional amino acid sequence variants are those in which one or more amino acid residues are introduced into a predetermined site in a subject peptide although random insertion is also possible with suitable screening of the resulting product. Deletional variants may be made by removing one or more amino acids from the sequence of a subject peptide. Substitutional amino acid variants are those in which at least one residue in the sequence has been removed and a different residue inserted in its place. Typical substitutions are those made in accordance with the following Table 1:

TABLE 1
Suitable residues for amino acid substitutions

| Original Residue | Exemplary Substitutions |
|---|---|
| Ala (A) | Ser |
| Arg (R) | Lys |
| Asn (N) | Gln; His |
| Asp (D) | Glu |
| Cys (C) | Ser |
| Gln (Q) | Asn |
| Glu (E) | Asp |
| Gly (G) | Pro |
| His (H) | Asn; Gln |
| Ile (I) | Leu; Val |
| Leu (L) | Ile; Val |
| Lys (K) | Arg; Gln; Glu |
| Met (M) | Leu; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

When the synthetic peptide is derivatised by amino acid substitution, the amino acids are generally replaced by other amino acids having like properties such as hydrophobicity, hydrophilicity, electronegativety, bulky side chains and the like. As used herein, the terms “derivative”, “analogue”, “fragment”, “portion” and “like molecule” refer to a subject peptide having the amino acid sequence as set forth in SEQ ID NOs:1, 2, 3, or 4, having an amino acid substitution, insertion, addition, or deletion, as long as said derivative, analogue, fragment, portion, or like molecule retains the ability to enter and selectively kill transformed or neoplastic cells.

The synthetic peptides of the present invention may be synthesized by a number of known techniques. For example, the peptides may be prepared using the solid-phase technique initially described by Merrifield (1963) in J. Am. Chem. Soc. 85:2149-2154. Other peptide synthesis techniques may be found in M. Bodanszky et al. Peptide Synthesis, John Wiley and Sons, 2d Ed., (1976) and other references readily available to those skilled in the art. A summary of polypeptide synthesis techniques may be found in J. Sturart and J. S. Young, Solid Phase Peptide Synthesis, Pierce Chemical Company, Rockford, Ill., (1984). Peptides may also be synthesized by solution methods as described in The Proteins, Vol. 1, 3d Ed., Neurath, H. et al., Eds., pp. 105-237, Academic Press, New York, N.Y. (1976). Appropriate protective groups for use in different peptide syntheses are described in the texts listed above as well as in J. F. W. McOmie, Protective Groups in Organic Chemistry, Plenum Press, New York, N.Y. (1973). The peptides of the present invention may also be prepared by chemical or enzymatic cleavage from larger portions of the p53 protein or from the full length p53 protein. Likewise, leader sequences for use in the synthetic peptides of the present invention may be prepared by chemical or enzymatic cleavage from larger portions or the full length proteins from which such leader sequences are derived.

Additionally, the peptides of the present invention may also be prepared by recombinant DNA techniques. For most amino acids used to build proteins, more than one coding nucleotide triplet (codon) can code for a particular amino acid residue. This property of the genetic code is known as redundancy. Therefore, a number of different nucleotide sequences may code for a particular subject peptide selectively lethal to malignant and transformed mammalian cells. The present invention also contemplates a deoxyribonucleic acid (DNA) molecule that defines a gene coding for, i.e., capable of expressing a subject peptide or a chimeric peptide from which a peptide of the present invention may be enzymatically or chemically cleaved.

A subject peptide having one or more amino acids in the D-form may be synthesized by incorporating into the peptide chain, one or more D-amino acids instead of the naturally occurring L-amino acids. The synthesis of linear D-form peptides may be prepared using conventional protocols of peptide synthesis including synthesis by automated procedure. See, e.g., Scheibler, L. and Chorev, J. (2003) In Synthesis of Peptides and Peptidomimetrics (Houben-Weyl Methods of Organic Chemistry, 4^{th} Ed., Vol. 22C)(Goodman, M., ed), pp 528-551, Thieme, Stuttgart.

The reduced isostere pseudopeptide bond is a pseudopeptide bond which enhances stability to proteolytic cleavage with little or no loss of biological activity. Thus a subject peptide may be identical to an L-amino acid peptide having the amino acid sequences set forth in any of SEQ ID NOs: 1-3 (p53 peptides) or any of SEQ ID NOs: 4-24 or 26-27 (membrane penetrating leader sequences) except that one or more peptide bonds are replaced by an isostere pseudopeptide bond. Methods of synthesizing peptides with one or more reduced isostere pseudopeptide bonds are well known in the art. See Couder et al. (1993) Int. J. Peptide Protein Res. 41:181-184, incorporated by reference herein as if fully set forth.

Peptide bonds may also be replaced with retro-inverso pseudopeptide bonds. Thus a subject peptide may be identical to the L-amino acid peptides having the amino acid sequences set forth in any of SEQ ID NOs: 1-3 (p53 peptides) or any of SEQ ID NOs: 4-24 or 26-27 (membrane penetrating leader sequences) except that one or more retro-inverso pseudopeptide bonds are substituted for peptide bonds. Procedures for synthesizing peptides with one or more retro-inverso pseudopeptide bonds are available in the literature extant; see e.g., Dalpozzo, et al. (1993) In. J. Peptide Protein Res. 41:561-566, incorporated by reference herein as if fully set forth.

A subject RI peptide can be synthesized using D-amino acids and attaching the amino acids in a peptide chain such that the sequence of amino acids in the retro-inverso peptide analog is the exact opposite of that in the selected peptide which serves as the model. Thus, the retro-inverso peptide of the peptide set forth in SEQ ID NO:1 comprises all D-amino acids assembled in the following sequence: LLKWLDSFTEQSLPP (SEQ ID NO:28). The retro-inverso peptide of the peptide set forth in SEQ ID NO:2 comprises all D-amino acids assembled in the following sequence: SFTEQSLPP (SEQ ID NO:29). The retro-inverso peptide of the peptide set forth in SEQ ID NO:3 comprises all D-amino acids assembled in the following sequence: LLKWLDSFTE (SEQ ID NO:30).

The retro-inverso peptide may be synthesized by Fmoc chemistry on a Fmoc-2,4-dimethyloxy-4′(carboxymethyloxy)-benzhydrylamine resin. See, e.g., Briand, J. P., et al., (1995) “Retro-Inverso peptidomimetics as a new immunological probe: validation and application to the detection of autoantibodies in rheumatic diseases”. J. Biol. Chem. 270, 11921-11926, which is incorporated by reference herein as if fully set forth. The NH₂-termini of the retro-inverso peptides can be acetylated. After acid cleavage, the crude peptides can be purified by standard methods such as on a column chromatography using a preparative HPLC apparatus. The purity of the retro-inverso peptides can be determined by analytical HPLC or other well-known methods known in the art.

The appropriate stereoisomers of L-Ile and L-Thr in RI peptides (and RI sequences within PRMI peptides) are D-alloIle and D-alloThr because of the presence of two chiral centers in these amino acids.

With respect to synthesis of a subject PMRI-peptide, solution-based methodologies are preferred. Solution-based chemistry can generate suitably protected gem-diaminoalkyl and 2-alkylmalonyl moieties needed for the synthesis of PMRI-peptides by a variety of well-known reactions. The generated crude building blocks and the pseudopeptide units may then be subjected to purification and characterization.

PMRI-peptides may also be made by solid phase synthesis either by incorporation of precursors such as HO-Ala-(RS)-mPhe-(R)-Lys(N²-Boc)-NH₂ or HO-mGly-(R)-Phe-NH₂ to generate the PMRI unit on resin, or by incorporation of the preformed PMRI unit PG-Xaa¹_{ψ}[NHCO]Xaa²—OH (ψ=pseudopeptide bond) as a building block. However, slow reaction rates, side reactions, and lack of compatibility between reaction conditions and the solid support complicate the solid-phase synthesis of PMRI-peptides and prevent it from becoming the method of choice. See, e.g., Scheibler, L. and Chorev, M. (2003) In Synthesis of Peptides and Peptidomimetics (Houben-Weyl, Methods of Organic Chemistry, 4^{th} Ed., Vol. 22C) (Goodman, M., ed), pp. 528-551, Thieme, Stuttgart. The disclosure of all patents, papers, and book chapters cited herein, are incorporated by reference herein as if fully set forth.

When applied to cells grown in culture, synthetic peptides are selectively lethal to malignant or transformed cells, resulting in dose dependent reduction in cell number. The effect is observable generally within two to three and at most 48 hours. A line of rat pancreatic acinar cells (BMRPA.430) grown in culture was transformed with K-ras. The normal cell line displays the architecture typical of pancreatic acinar cells; the transformed cells (TUC-3) lack the differentiated morphology of acinar cells, appearing as typical pancreatic cancer cells. When BMRPA.430 cells were treated with a synthetic peptide with the primary structure of SEQ ID NO:1 coupled to leader sequence SEQ ID NO:4, at a dosage of 50 μg/ml, the cells were not affected. However, when TUC-3 cells were treated with a peptide with the primary structure of SEQ ID NO:1 coupled to leader sequence SEQ ID NO:4, at a dosage of 100 μg/ml, they died within three to four days. Similar results were obtained when the same experiment was performed but SEQ ID NO:1 was substituted with either SEQ ID NO:2, or SEQ ID NO:3. Additionally, transformed and malignant cell death was observed in human breast carcinoma cell lines and Melanoma and HeLa cells treated with a synthetic peptide with the primary structure of SEQ ID NO:1 coupled to leader sequence SEQ ID NO:4, at a dosage of 100 μg/ml. In contrast, the same synthetic peptide at the same dosage had no effect on non-malignant and non-transformed human breast or fibroblast cell lines.

When the leader sequence set forth in SEQ ID NO:4 was positioned at the carboxy terminal end of PNC29, a control protein having the following amino acid sequence: MPFSTGKRIMLGE (SEQ ID NO: 25), there was no effect on malignant or normal cells.

Additionally, the peptide having the amino acid sequence as set forth in SEQ ID NO:3 fused at the carboxy terminal end to the leader peptide set forth in SEQ ID NO:4, has no effect on the ability of human stem cells to differentiate into hematopoietic cell lines in the presence of growth factors. This indicates that this peptide will not be injurious to bone marrow cells when administered as a chemotherapeutic agent. See Kanovsky et al., (Oct. 23, 2001) Proc. Nat. Acad. Sci. USA 98(22); 12438-12443, the disclosure of which is incorporated by reference herein as if fully set forth.

When cultured cancer cells were treated with a peptide with the primary structure of SEQ ID NO:1 without a leader sequence attached, at a dosage of 100 μg/ml, the cells were unaffected. Similarly, when cultured cancer cells were treated with leader sequence SEQ ID NO:4, the presently preferred leader sequence, at the same dosage, the cell were also unaffected. These results indicate that the leader sequence of the synthetic peptide allows the synthetic peptide to cross the cellular membranes of treated cells and that the effect of the synthetic peptide is necessarily intracellular.

In order to determine whether the synthetic peptides acted by interfering with the binding of the p53 protein and the MDM-2 protein, the synthetic peptides were tested on transformed colorectal adenocarcinoma cells that had been rendered incapable of making the p53 protein by homozygous deletion. Surprisingly, the synthetic peptides selectively killed the transformed cells, but had no effect on the normal cells. These results indicate that the mechanism of action appears to be independent of the p53 protein binding to the MDM-2 protein, as the p53 peptide selectively kills transformed cells that do not produce the p53 protein at all. These results indicate that interference with binding of the p53 protein to the MDM-2 protein may not be the mechanism by which synthetic peptides cause selective death of malignant and transformed cells. Although the synthetic peptides disclosed herein, their derivatives, analogues, and peptidomimetic molecules are useful in the treatment of neoplastic disease such as cancer, the mechanism for action on transformed and malignant cells has not been discovered.

The peptides of the present invention are effective against neoplastic cells in vivo. For example, mice having been xenotransplanted with the pancreatic carcinoma cells BMRPA1.TUC-3 and having developed tumor size of about 3-6 mm, have the size of such tumors drastically reduced after administration of a subject synthetic peptide, e.g., a peptide having the amino acid sequence as set forth in SEQ ID NO:3 fused to a leader sequence at the carboxy terminal end.

Consistent with the observed properties of the peptides of the invention, the subject peptides may be used to selectively kill neoplastic or malignant cells, i.e., cancer cells in animals, preferentially humans. The synthetic peptides of the present invention are thus administered in an effective amount to kill neoplastic cells in a subject animal or human.

The synthetic peptides of the present invention may be administered preferably to a human patient as a pharmaceutical composition containing a therapeutically effective dose of at least one synthetic peptide according to the present invention together with a pharmaceutical acceptable carrier. The term “therapeutically effective amount” or “pharmaceutically effective amount” means the dose needed to produce in an individual, suppressed growth including selective killing of neoplastic or malignant cells, i.e., cancer cells.

Preferably, compositions containing one or more of the synthetic peptides of the present invention are administered intravenously for the purpose of selectively killing neoplastic cells, and therefore, treating neoplastic or malignant disease such as cancer. Examples of different cancers which may be effectively treated using one or more the peptides of the present invention include but are not limited to: breast cancer, prostate cancer, lung cancer, cervical cancer, colon cancer, melanoma, pancreatic cancer and all solid tissue tumors (epithelial cell tumors) and cancers of the blood including but not limited to lymphomas and leukemias.

Administration of the synthetic peptides of the present invention may be by oral, intravenous, intranasal, suppository, intraperitoneal, intramuscular, intradermal or subcutaneous administration or by infusion or implantation. When administered in such manner, the synthetic peptides of the present invention may be combined with other ingredients, such as carriers and/or adjuvants. There are no limitations on the nature of the other ingredients, except that they must be pharmaceutically acceptable, efficacious for their intended administration, cannot degrade the activity of the active ingredients of the compositions, and cannot impede importation of a subject peptide into a cell. The peptide compositions may also be impregnated into transdermal patches, or contained in subcutaneous inserts, preferably in a liquid or semi-liquid form which patch or insert time-releases therapeutically effective amounts of one or more of the subject synthetic peptides.

The pharmaceutical forms suitable for injection include sterile aqueous solutions or dispersions and sterile powders for the extemporaneous preparation of sterile injectable solutions or dispersions. The ultimate solution form in all cases must be sterile and fluid. Typical carriers include a solvent or dispersion medium containing, e.g., water buffered aqueous solutions, i.e., biocompatible buffers, ethanol, polyols such as glycerol, propylene glycol, polyethylene glycol, suitable mixtures thereof, surfactants or vegetable oils. Sterilization may be accomplished utilizing any art-recognized technique, including but not limited to filtration or addition of antibacterial or antifungal agents. Examples of such agents include paraben, chlorbutanol, phenol, sorbic acid or thimerosal. Isotonic agents such as sugars or sodium chloride may also be incorporated into the subject compositions.

As used herein, a “pharmaceutically acceptable carrier” includes any and all solvents, dispersion media, coatings, antibacterial and antifungal agents, isotonic agents and the like. The use of such media and agents are well-known in the art.

Production of sterile injectable solutions containing the subject synthetic peptides is accomplished by incorporating one or more of the subject synthetic peptides described hereinabove in the required amount in the appropriate solvent with one or more of the various ingredients enumerated above, as required, followed by sterilization, preferably filter sterilization. In order to obtain a sterile powder, the above solutions are vacuum-dried or freeze-dried as necessary.

Inert diluents and/or assimilable edible carriers and the like may be part of the pharmaceutical compositions when the peptides are administered orally. The pharmaceutical compositions may be in hard or soft shell gelatin capsules, be compressed into tablets, or may be in an elixir, suspension, syrup or the like.

The subject synthetic peptides are thus compounded for convenient and effective administration in pharmaceutically effective amounts with a suitable pharmaceutically acceptable carrier in a therapeutically effective dosage. Examples of a pharmaceutically effective amount includes peptide concentrations in the range from about at least about 25 ug/ml to at least about 300 ug/ml.

A precise therapeutically effective amount of synthetic peptide to be used in the methods of the invention applied to humans cannot be stated due to variations in stage of neoplastic disease, tumor size and aggressiveness, the presence or extent of metastasis, etc. In addition, an individual's weight, gender, and overall health must be considered and will effect dosage. It can be generally stated, however, that the synthetic peptides of the present invention be administered in an amount of at least about 10 mg per dose, more preferably in an amount up to about 1000 mg per dose. Since the peptide compositions of the present invention will eventually be cleared from the bloodstream, re-administration of the pharmaceutical compositions is indicated and preferred.

The synthetic peptides of the present invention may be administered in a manner compatible with the dosage formulation and in such an amount as will be therapeutically effective. Systemic dosages depend on the age, weight, and condition of the patient and the administration route. An exemplary suitable dose for the administration to adult humans ranges from about 0.1 to about 20 mg per kilogram of body weight. Preferably, the dose is from about 0.1 to about 10 mg per kilogram of body weight.

In accordance with the present invention, there is also provided a method of treating neoplastic disease. The method comprises administering to a subject in need of such treatment, a therapeutically effective amount of a synthetic peptide hereinbefore described, including analogs and derivatives thereof. Thus for example, in one embodiment, an effective amount of a peptide comprising at least about six contiguous amino acids as set forth in SEQ ID NO:1 or an analog or derivative thereof fused on its carboxy terminal end to a leader sequence may be administered to a subject. In another embodiment, an effective amount of a peptide comprising at least from about eight (8) to at least about ten (10) contiguous amino acids as set forth in SEQ ID NO:1 or an analog or derivative thereof, fused on its carboxy terminal end to a leader sequence, may be administered to a subject. For example, an effective amount of a peptide having the amino acid sequence as set forth in SEQ ID NO:1 or an analog or derivative thereof, fused on its carboxy terminal end to a leader sequence may be administered to a subject. An effective amount of a peptide having the amino acid sequence as set forth in SEQ ID NO:2 or an analog or derivative thereof, fused on its carboxy terminal end to a leader sequence may also be administered to a subject. In still another embodiment, an effective amount of a peptide having the amino acid sequence set forth in SEQ ID NO:3 or an analog or derivative thereof, fused on its carboxy terminal end to a leader sequence may be administered to a subject. Any of the subject peptides comprising one or more D-amino acids, isostere pseudopeptide or retro-inverso pseudopeptide bonds, or any of the RI or PMRI peptides hereinbefore described and fused at the carboxy terminal end to a membrane-penetrating leader sequence, may also be used in a method of killing malignant or neoplastic cells in a patient.

In accordance with a method of treatment, a mixture of synthetic peptides may be administered. Thus, for example, in addition to administering one of the peptides, or analogs or derivatives thereof hereinbefore described in an effective amount, mixtures of two or more peptides or analogs or derivatives hereinbefore described may be administered to a subject.

Also provided by the present invention is a method of assessing the level of effectiveness of a peptide in selectively killing malignant, neoplastic, or transformed cells in vitro. The method comprises the steps of contacting malignant, transformed, or neoplastic cells with any of the peptides hereinbefore described, assessing the level of effectiveness based on the ratio or percentage of dead cells compared to live cells and evaluating the effects of the peptide on the growth of untransformed (normal) cells in culture. Thus, those peptides which kill malignant, transformed or neoplastic cells in vitro while exerting no negative effects on untransformed or normal cells in culture, would be considered valuable candidates for use in treating patients suffering from neoplastic disease.

The following examples further illustrate the invention and are not meant to limit the scope thereof.

### EXAMPLE I

The following experiment was performed to compare effectiveness of subject peptides having the leader sequence attached to the amino terminal end. As described supra, peptides synthesized with a leader sequence on the carboxyl terminal promoted α-helix formation in the peptide, which is the active conformation of the p53 part of this peptide when bound to MDM-2. As described supra, subject peptides having the amino acid sequences as set forth in SEQ ID NOs:1, 2, and 3 are strongly toxic to a wide variety of human cancer cells, including those that are homozygously p53 gene-deleted. An α-helix probability profile for each peptide having the sequences set forth in SEQ ID NOs:1-3 was performed using two different methods, one using helix probabilities from the protein database (Karplus, K. et al., (1998) Bioinformatics 14:846-856), and the other using the Ising model based on helix nucleation (σ) and growth (s), equilibrium constants determined experimentally from block copolymers for each of the twenty naturally occurring L amino acids, modified by inclusion of the effects of charges on these parameters as described in Vasquez, M., et al. (1987) Biopolymers 26:351-372 and Vasquez, M., et al., (1987) Biopolymers 26:373-393. Probability profiles indicated that if the leader sequence is on the amino terminal end, even though the peptide still transverses the cell membrane, the α-helical content is much lower.

The peptide having the sequence set forth in SEQ ID NO:3 was synthesized by solid phase synthesis with the leader sequence attached to the amino terminal end. This peptide is labeled PNC28′ in Table 2 below. The PNC28′ peptide was incubated with transformed pancreatic cancer (TUC-3) cells at three different concentrations, i.e., 25, 50 and 100 μg/ml. After two weeks of incubation, at the highest dose of peptide, there was no cell death, and approximately half of the cells were seen to form acini and exhibited the untransformed morphological phenotype. The same phenomena were observed at 50 μg/ml, and at 25 μg/ml significantly fewer cells were seen to revert. In contrast, when the leader sequence was attached to the carboxyl terminal end of the peptide (PNC28 in Table 2), at dosages of 50 and 100 μg/ml. 100% cell death occurred in about 4 days.

These results show that the leader sequence is preferentially added to the carboxyl terminal end of the MDM-2 portion of the p53 peptide to enable the peptide to cross the cell membrane and specifically kill malignant cells. In Table 2, the leader sequence is KKWKMRRNQFWVKVQRG (SEQ ID NO:4).

TABLE 2

| | | | |
|---|---|---|---|
| NAME | p53 seq. | PEPTIDE | EFFECT |

| | | | |
|---|---|---|---|
| 1. PNC 21 | 12-20 | (PPLSQETFS) | Cytotoxic |
| | | (SEQ ID NO:2)- Leader | |
| 2. PNC 27 | 12-26 | (PPLSQETFSDLWKLL) | Cytotoxic |
| | | (SEQ ID NO:1)- Leader | |
| 3. PNC 28 | 17-26 | (ETFSDLWKLL) | Cytotoxic |
| | | (SEQ ID NO:3)- Leader | |
| 4. PNC 28′ | 17-26 | Leader (ETFSDLWKLL) | No cell |
| | | (SEQ ID NO:3) | death and |
| | | | reversion |

These results indicate the uniqueness of the subject peptides. i.e., the leader or cluster of positively charged residues must be placed at the carboxy terminal end of any effector peptide for cancer cell toxicity.

### EXAMPLE II

Nu/Nu mice (Harlan Laboratories, Indianapolis, Ind., n=10) and weighing 20-22 g, were xenotransplanted subcutaneously (s.c.) with live pancreatic carcinoma cells BMRPA1.TUC-3 (1×10⁶ cells/mouse) in the left hind region. Tumors were allowed to develop and grow and during daily examinations it was observed that all mice developed tumors with very similar growth rates.

After 12 days the tumors had reached sizes of 3 to 6 mm diameter and the mice were separated into two groups of 5 mice each. Each group was implanted s.c. with Alzet® osmotic pumps to deliver in a constant rate and over a defined period of 14 days a total volume of 0.095 ml volume of normal saline containing the respective peptide at a concentration of 20 mg/mouse. One group of mice received PNC-28 (the peptide having the amino acid set forth in SEQ ID NO:3) fused at its carboxy terminal end to the penetratin leader sequence (SEQ ID NO:4) and the other group of mice received PNC-29, a control peptide of similar size, having the following amino acid sequence: MPFSTGKRIMLGE (SEQ ID NO: 25). The pumps were filled according to the manufacturers guidelines and under sterile conditions. The pumps were implanted s.c. on the left flank of the anaesthetized mice by creating a pocket underneath the mouse skin into which the tiny pumps were inserted. Each pocket was closed with a simple suture. From their inside chamber the pumps delivered continuously 0.25 μl/hr into each mouse. The mice were observed until they had recovered from the surgery when they were returned to the isolation ward of the animal facility. Since the animals were Nu/Nu mice and, thus, immuno-compromised they are highly susceptible when exposed to pathogens. Surgery and all preceding and post-surgical treatments were therefore performed in a sterile hood environment.

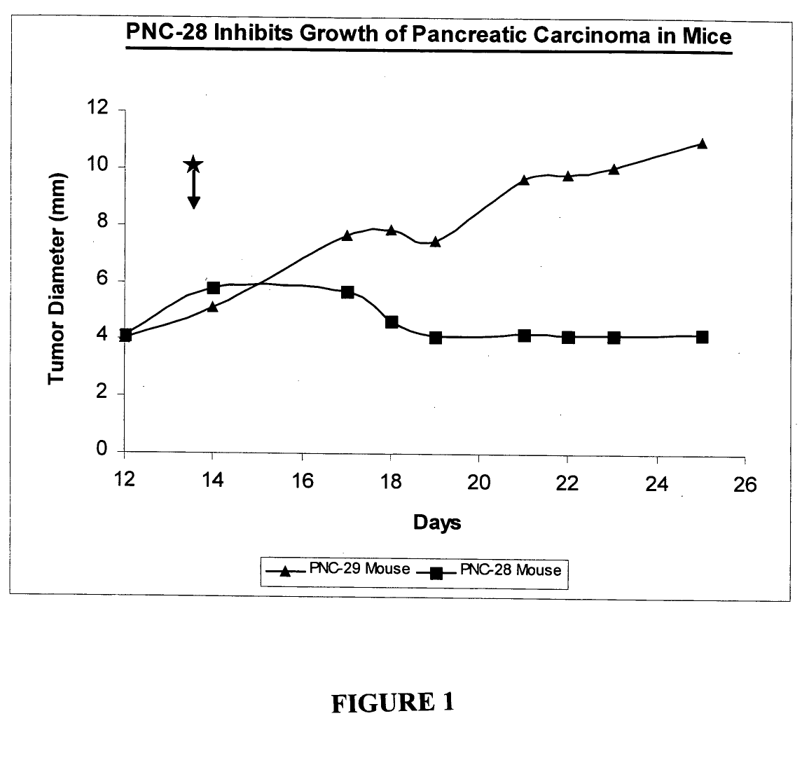

As shown clearly in FIG. 1, PNC-28 within a 48 to 72 hr period of delivery into the mouse effectively arrests tumor growth. In contrast, the control peptide PNC-29 had no effect on normal or tumor cells. In PNC29-treated mice, tumors kept growing at a continuous rate resulting in tumors of 10 to 16 mm diameter over the 2-week treatment and follow-up period when the pumps cease to release any more peptide solution. Statistical analyses of the measurement of tumor size in both groups of mice has produced a significance between them of p<0.001.

### EXAMPLE III

Using the same methodology of Example II, pumps were started at the same time as live pancreatic carcinoma cells BMRPA1.TUC-3 (1×10⁶ cells/mouse) were xenotransplanted into mice (n=10). Five mice were administered PNC28 and 5 mice were not treated at all (sham treated). Results are tabulated below.

TABLE 3

| | 7 Days | 14 Days | 21 Days |
|---|---|---|---|
| Treatment | Tumor Size | | |
| Sham treated | 4.8 ± 1.8 | 11.7 ± 2.3 | 14.8 ± 3.6 |
| PNC-28 treated | 3 ± .6 | 3.1 ± .9 | 4.4 ± .8 |

### EXAMPLE IV

Using the same methodology as described in Example II, live pancreatic carcinoma cells BMRPA1.TUC-3 (1×10⁶ cells/mouse) were transplanted to the peritoneal cavity of five mice. Pumps were placed in the right shoulder region at the same time of tumor cell transplantation. In all five mice, there were no visible tumors after three weeks.

### EXAMPLE V

A peptide having an amino acid sequence as set forth in SEQ ID NO:2 or 3 is synthesized with one or more amino acids in the D-form by solid phase synthesis with a membrane-penetrating leader sequence attached to the carboxy terminal end. The solid-phase peptide synthesis methodology involves coupling each protected amino acid residue to a resin support, preferably a 4-methyl-benzhydrylamine resin, by activation with dicyclohexylcarbodimide to yield a peptide with a C-terminal amide. Side-chain functional groups are protected as follows: benzyl for serine, threonine, glutamic acid, and aspartic acid; tosyl for histidine and arginine; 2-chlorobenzyloxycarbonyl for lysine and 2,6-dichlorobenzyl for tyrosine. Following coupling, the t-butyloxycarbonyl protecting group on the alpha amino function of the added amino acid is removed by treatment with trifluoroacetic acid followed by neutralization with di-isopropyl-ethylamine. The next protected residue is then coupled onto the free amino group, propagating the peptide chain. After the last residue has been attached, the protected peptide-resin is treated with hydrogen fluoride to cleave the peptide from the resin, as well as deprotect the side chain functional groups. Crude product can be further purified by reverse phase HPLC. The peptide may be incubated with malignant, transformed or neoplastic cells such as pancreatic cancer cells (TUC3) at three different concentrations, i.e., 25, 50, and 100 μl/ml, in order to assess the level of effectiveness in killing such cells at these concentrations.

### EXAMPLE VI

A retro-inverso (RI) peptide having all D-amino acids assembled in the reverse order of the amino acid sequence set forth in SEQ ID NO: 2 or SEQ ID NO: 3 is synthesized using D-amino acids. The retro-inverso form is synthesized by standard Fmoc chemistry on an ABI 433A Peptide Synthesizer (Applied Biosystems, Foster City, Calif., United States). See, Ben-Yedida, et al., (2002) Molecular Immunology, 39:323-331. Crude product is further purified by reverse-phase HPLC over a C18 preparatory column (Varian, Palo Alto, Calif., United States). The identity of the peptides is confirmed by mass spectrometry. The peptide is fused to a membrane-penetrating leader sequence at its carboxy terminal end and may be incubated with malignant, transformed or neoplastic cells such as pancreatic cancer cells (TUC3) at three different concentrations, i.e., 25, 50, and 100 μl/ml, in order to assess the level of effectiveness in killing such cells at these concentrations.

### EXAMPLE VII

A partially modified retro-inverso (PMRI) peptide having a portion but not all of the amino acids in D form and in reverse order to the sequence set forth in SEQ ID Nos:2 or 3 is synthesized using solution-based chemistry to generate suitably protected gem-diaminoalkyl and 2-alkylmalonyl moieties needed for the synthesis of PMRI-peptides. See, e.g., Scheibler, L. and Chorev, M. (2003) In Synthesis of Peptides and Peptidomimetics (Houben-Weyl) Methods of Organic Chemistry, 4^{th} Ed., Vol. 22C) (Goodman, M., ed), pp. 528-551, Thieme, Stuttgart. The Curtius and Hofmann rearrangements, of acyl azides and acyl amides are utilized for the synthesis of PMRI-peptides. The migrating group retains its configuration during rearrangement, offering a means for the conversion of optically pure amino acids into the topographically complementary gem-diaminoalkyl derivatives. The isocyanate intermediates are trapped in a Goldschmidt-Wick-type reaction with an excess of carboxylic acid to afford adducts with the N,N′-diacylated gem-diaminoalkyl residue. Trapping the isocyanate with an N-protected amino acid affords the retro-inverso pseudopeptide unit. The generated crude building blocks and the pseudopeptide units are subjected to purification and characterization. The peptide is fused at its carboxy terminus to a membrane-penetrating leader sequence and may be incubated with malignant, transformed or neoplastic cells such as pancreatic cancer cells (TUC3) at three different concentrations, i.e., 25, 50, and 100 μl/ml, in order to assess the level of effectiveness in killing such cells at these concentrations.

The foregoing specification, and the experimental results reported therein are illustrative and are not limitations of the scope of applicant's invention. Those skilled in the art will appreciate that various modifications can be made without departing from applicant's invention.

1. A peptide comprising at least six contiguous amino acids of the amino acid sequence: PPLSQETFSDLWKLL (SEQ ID NO: 1), or an analog or derivative thereof, wherein said peptide or analog or derivative thereof is fused at its carboxy-terminal end to a membrane-penetrating leader sequence, is selectively lethal to malignant or transformed cells, and wherein at least one amino acid is a D-amino acid or wherein at least one peptide bond is replaced with an isostere pseudopeptide bond or a retro-inverso pseudopeptide bond.

2. A retro-inverso (RI) peptide comprising at least six contiguous D-amino acids assembled in the reverse order of the amino acid sequence: PPLSQETFSDLWKLL (SEQ ID NO: 1), or an analog or derivative thereof, wherein said RI peptide is fused at its carboxy-terminal end to a membrane-penetrating leader sequence and wherein said peptide is selectively lethal to malignant or transformed cells.

3. A partially modified retro-inverso (PMRI) peptide comprising at least six contiguous amino acids wherein a portion of the at least six amino acids are D-amino acids assembled in reverse order of the amino acid sequence set forth in SEQ ID NO:1, or an analog or derivative thereof, wherein said PMRI peptide is fused at its carboxy-terminal end to a membrane-penetrating leader sequence and is selectively lethal to malignant or transformed cells.

4. The peptide of claim 1 comprising the amino acid sequence: PPLSQETFS (SEQ ID NO: 2) or ETFSDLWKLL (SEQ ID NO: 3), or an analog or derivative thereof.

5. The peptide of claim 2 comprising at least six contiguous D-amino acids assembled in the reverse order of the amino acid sequence PPLSQETFS (SEQ ID NO: 2) or ETFSDLWKLL (SEQ ID NO: 3), or an analog or derivative thereof.

6. The peptide of claim 3 wherein a portion of the at least six D-amino acids are assembled in reverse order of the sequence PPLSQETFS (SEQ ID NO: 2) or ETFSDLWKLL (SEQ ID NO: 3), or an analog or derivative thereof.

7. The peptide, analog or derivative thereof according to claim 1 wherein the N-terminal amino acid of the peptide comprises a D-amino acid.

8. The peptide, analog or derivative thereof according to claim 1 wherein the carboxy-terminal amino acid of the membrane-penetrating leader sequence comprises a D-amino acid.

9. The peptide of claim 1 wherein the most N terminal peptide bond of the peptide is replaced with an isostere pseudopeptide bond or a retro-inverso pseudopeptide bond.

10. The peptide, analog or derivative thereof according to any of claims 1-9, wherein the membrane-penetrating leader sequence is at least one of penetratin (SEQ ID NO:4), Arg₈ (SEQ ID NO: 26), a poly-R having the amino acid sequence set forth in SEQ ID NO: 27, TAT of HIV1 (SEQ ID NO: 5), D-TAT (SEQ ID NO: 6), R-TAT (SEQ ID NO: 7), SV40-NLS (SEQ ID NO:8), nucleoplasmin-NLS (SEQ ID NO: 9), HIV REV (SEQ ID NO: 10), FHV coat (SEQ ID NO: 11), BMV GAG (SEQ ID NO: 12), HTLV-II (REX) (SEQ ID NO: 13), CCMV GAG (SEQ ID NO: 14), P22N (SEQ ID NO: 15), Lambda N (SEQ ID NO:16), Phi N (SEQ ID NO:17), yeast PRP6 (SEQ ID NO:18), human U2AF (SEQ ID NO:19), human C—FOS (SEQ ID NO:20), human C-JUN (SEQ ID NO:21), yeast GCN4 (SEQ ID NO:22), KLALKLALKALKAALKLA (SEQ ID NO:23), or p-vec (SEQ ID NO:24).

11. The peptide, analog, or derivative thereof according to claim 10 wherein the membrane-penetrating leader sequence comprises at least one D-amino acid or wherein at least one peptide bond is replaced with an isostere pseudopeptide bond or a retro-inverso pseudopeptide bond.

12. The peptide, analog, or derivative thereof according to claim 11 wherein the membrane-penetrating leader sequence comprises all D-amino acids assembled in reverse order to any of the sequences set forth in SEQ ID NOs: 4-24 or SEQ ID NOs:26-27.

13. The peptide, analog, or derivative thereof according to claim 11 wherein the membrane-penetrating leader sequence comprises a portion of D-amino acids assembled in reverse order to any of the sequences set forth in SEQ ID NOs: 4-24 or SEQ ID NOs: 26-27.

14. A pharmaceutical composition comprising at least one peptide, analog or derivative thereof according to claims 1-9 admixed with a pharmaceutically acceptable carrier.

15. A pharmaceutical composition comprising at least one peptide, analog or derivative thereof according to claim 10 admixed with a pharmaceutically acceptable carrier.

16. A pharmaceutical composition comprising at least one peptide, analog, or derivative thereof according to claim 11 admixed with a pharmaceutically acceptable carrier.

17. A pharmaceutical composition comprising at least one peptide, analog, or derivative thereof according to claim 12 admixed with a pharmaceutically acceptable carrier.

18. A pharmaceutical composition comprising at least one peptide, analog, or derivative thereof according to claim 13 admixed with a pharmaceutically acceptable carrier.

19. A method of selectively killing malignant or neoplastic cells in a subject, said method comprising administering to the subject, a therapeutically effective amount of at least one peptide of claims 1-9 or an analog or derivative thereof.

20. A method of selectively killing malignant or neoplastic cells in a subject, said method comprising administering to the subject, a therapeutically effective amount of at least one peptide of claim 10 or an analog or derivative thereof.

21. A method of selectively killing malignant or neoplastic cells in a subject, said method comprising administering to the subject, a therapeutically effective amount of at least one peptide of claim 11 or an analog or derivative thereof.

22. A method of selectively killing malignant or neoplastic cells in a subject, said method comprising administering to the subject, a therapeutically effective amount of at least one peptide of claim 12 or an analog or derivative thereof.

23. A method of selectively killing malignant or neoplastic cells in a subject, said method comprising administering to the subject, a therapeutically effective amount of at least one peptide of claim 13 or an analog or derivative thereof.

24. A method of assessing the level of effectiveness of a peptide in selectively killing malignant, transformed or neoplastic cells, the method comprising: contacting malignant, transformed or neoplastic cells in vitro with a peptide of any of claims 1-9, or an analog or derivative thereof, and assessing the level of effectiveness of the peptide based on the ratio or percentage of dead cells compared to live cells and its effect on the growth of untransformed cells in culture.

25. A method of assessing the level of effectiveness of a peptide in selectively killing malignant, transformed, or neoplastic cells, the method comprising: contacting malignant, transformed or neoplastic cells in vitro with a peptide of claim 10, or an analog or derivative thereof, and assessing the level of effectiveness of the peptide based on the ratio or percentage of dead cells compared to live cells and its effect on the growth of untransformed cells in culture.

26. A method of assessing the level of effectiveness of a peptide in selectively killing malignant, transformed, or neoplastic cells, the method comprising: contacting malignant, transformed or neoplastic cells in vitro with a peptide of claim 11, or an analog or derivative thereof, and assessing the level of effectiveness of the peptide based on the ratio or percentage of dead cells compared to live cells and its effect on the growth of untransformed cells in culture.

27. A method of assessing the level of effectiveness of a peptide in selectively killing malignant, transformed, or neoplastic cells, the method comprising: contacting malignant, transformed or neoplastic cells in vitro with a peptide of claim 12, or an analog or derivative thereof, and assessing the level of effectiveness of the peptide based on the ratio or percentage of dead cells compared to live cells and its effect on the growth of untransformed cells in culture.

28. A method of assessing the level of effectiveness of a peptide in selectively killing malignant, transformed, or neoplastic cells, the method comprising: contacting malignant, transformed or neoplastic cells in vitro with a peptide of claim 13, or an analog or derivative thereof, and assessing the level of effectiveness of the peptide based on the ratio or percentage of dead cells compared to live cells and its effect on the growth of untransformed cells in culture.
